(19) United States
(12) Patent Application Publication
(10) Pub. No.: US 2009/0275816 A1
(43) Pub. Date: Nov. 5, 2009

(54) DEVICE CASING

(76) Inventors: Tuomas Planman, Espoo (FI); Jukka Planman, Helsinki (FI)

Correspondence Address: MCCORMICK, PAULDING & HUBER LLP, CITY PLACE II, 185 ASYLUM STREET, HARTFORD, CT, 06103, US

(21) Appl. No.: 12/374,173

(22) PCT Filed: Jul. 19, 2007
(86) PCT No.: PCT/FI07/50432

§ 371 Date: Feb. 10, 2009

(30) Foreign Application Priority Data
Jul. 19, 2006 (FI) 20060691

(51) Int. Cl. A61B 5/15; A61B 5/00

(52) U.S. Cl. 600/365; 600/309

(57) ABSTRACT

The invention relates to portable electronic devices, particularly health-monitoring devices, and their casings. Such a device comprises a first case part and a second case part, which are arranged in the first position to lie tightly against each other, in order to protect the internal parts of the device, so that a compression joint is formed between them. The case parts are also arranged to slide in one direction relative to each other to a second position, in order to reveal at least one user-interface element. According to the invention, the direction of the said joint differs over its entire length essentially from the said sliding direction. The invention permits the manufacture of health-monitoring devices that are more tightly sealed and user-friendly than previously.

### CROSS REFERENCE TO RELATED APPLICATIONS

This application is entitled to the benefit of and incorporates by reference essential subject matter disclosed in International Patent Application No. PCT/FI2007/050432 filed on Jul. 19, 2007 and Finnish Patent Application No. 20060691 filed Jul. 19, 2006.

### TECHNICAL FIELD

The present invention relates to portable body fluid measuring devices.

### BACKGROUND OF THE INVENTION

A case, device, or device case of this kind is arranged to open from its primary state, in order to effect the operations of the device, or, for example, in order to reveal its operational internal components or elements, such as user interfaces, or health-monitoring elements (or both).

Various kinds of opening and closing mechanisms are used in portable digital devices, mainly for two reasons. Firstly, it is wished to reduce the external dimensions of the device for transportation, whereas in use, on the other hand, there is often benefit from the larger dimensions of the opened position. Secondly, the devices' delicate components (displays, keys, etc.) need protection from the stresses imposed by the environment (for instance, scratching, dirtying, wetting) during transportation.

In mobile telephones, for example, three case solutions are mainly used: clamshell models (hinges), slider models (rails), and rotating models (two parts set on top of each other that rotate horizontally around a pivot). All of these have weaknesses.

The casing solutions of mobile telephones are primarily designed to serve the user of the device as a telephone, portable computer, or media player. Therefore they are not very suitable for personal health-monitoring devices, because the cleanliness, i.e. hygienic requirements of the latter are considerably greater. For example, the internal parts of a blood-sugar measuring device must not be easily exposed to dust and other impurities. A considerable number of diabetics measure their blood sugar several times a day and carry the device with them, so that there is a real danger of dirtying. In addition, because measurement is an intimate operation, it must be easy and discrete even in public places.

As far as is known, at present not a single device is presently commercially available, in which all the means required for personal blood-sugar measurement are encased in a well-sealed and practical manner. Existing solutions are mainly based on a separate meter, piercing device and test strips. Some current meters contain internal strip cassettes where strips are individually sealed (see e.g., U.S. Pat. No. 5,863,800). All of these are then transported in a carrying bag. However, such an equipment kit is difficult to use, thus making the kit not very suitable for regular personal health monitoring.

### BRIEF SUMMARY OF THE INVENTION

The invention is intended to reduce the defects of the state of the art disclosed above and create a new solution, which will permit the manufacture of better-sealed device casing. In particular, the invention is intended to create a new personal health-monitoring device, the important areas of which can be closed tightly and the use of which is easy and discrete.

The invention is based on the idea that, in the device, there is a first and a second case part, which are arranged to slide relative to each other and in the closed (first) position to press tightly against each other, so that at no point is the joint between them parallel to the direction of sliding, but is entirely a so-called butt joint (compression joint). In addition, the joint is continuous and forms a closed path that travels on the outer surface of the device.

Many advantages are gained with the aid of the invention. In the vicinity of the outer surface of the device, no rubbing movement at all appears when sliding, thus preventing particles from travelling into the inner parts of the device. In addition, a compressive force acts the entire area of the joint when pressing the case parts together in the sliding direction, allowing the joint to close tightly over its full length.

Thus the invention permits the device to be closed tightly, protecting not only the sensitive components, but also all parts of the opening mechanism. The mechanism permits the versatile use of the device, even with one hand, while it has no protruding parts, or any that restrict shaping, or hinges, or joints parallel to the sliding direction that may collect dirt.

We have found to be particularly advantageous an embodiment, in which the joint between the first and second case parts is clearly at a slant relative to the direction of movement of the parts, at least partly, and typically on at least two sides. This facilitates using the device with one hand while, when the parts are pressed together, the angled joint causes a force component at right-angles to the direction of movement, which can be utilized to achieve even tighter closing.

The opening movement is preferably linear. However, curved rails, for instance, and other generally known mechanisms and additional components can also be used in the movement, in order to achieve movement paths in different directions. The movement can also comprise several parts, for example, so that the first movement opens/closes the tight joint while one or more other movements can be implemented after this, for example, relating to the operating positions and operations of the device.

The operations produced in the device by a single or multiple-part opening movement can be advantageously effected by using various switches. For example, if the second opening movement normally sets measuring devices to stand by for operation, moving a switch can change the same movement, for example, to bring out equipment relating to the care of a disease, such as supplementary nutrition, or to permit the device to be opened for changing components.

By means of the solution according to the invention, the device can be conveniently opened and the operational internal part contained in the device easily pulled out. This operational inner part can be, for example, an interface element and/or a health-monitoring element or a measuring means. Examples of health-monitoring elements and measuring means are biological test strips and needle devices. These are, for example, strips and devices intended for measuring the sugar content of blood and a corresponding analyte (i.e. measurable quantity).

In one application, the interface elements of the device (such as buttons and displays and the other interface elements itemized below) are located on the outer surface of the device while the health-monitoring element or measurement means is located inside the device.

In a second preferred application, the health-monitoring device according to the invention comprises means for analysing a body fluid, or some other physiological, chemical, or biological component or property, such as blood, in which case the said means are arranged to be covered in the first position and to be exposed and preferably made ready for measurement with the aid of movement between the first and second case parts. Thus, the device can be used to measure from a person properties and analytes and parameters that are entirely freely selected. Opening the device can be used to reveal its operational internal part. This can be set to be ready for measurement after, or preferably in conjunction with opening.

There is particular benefit from the casing in connection with body fluid measuring devices that comprise elements sensitive to dust, dirt, moisture, or oxygen, such as circuit boards, or chemical or physical analysis means.

The casing of a blood-sugar meter or other health-monitoring device, particularly with a bevelled seam, permits measurements to be made very discretely, even using one hand. The sensitive part of the device, which also attracts attention, is inside the case parts, so that the device does not have the outward appearance of a measuring device.

According to one embodiment, the essential electrical operations of the device are located in an internal element, which is protected with the aid of two cover elements (case parts) placed against each other and around the internal element. The cover elements can be symmetrical and set against each other with the second one turned upside down and in an opposite position to the first one, in which case a rectangular case will be formed, for example. The bevelled dividing joint between the cover elements allows the device to be sealed when it is closed, because the joint is pressed tightly shut, unlike horizontal sliding joints. The bevelled sliding joint also permits the ergonomically agreeable operation of the device with one hand. The long flat surfaces of the cover elements and the internal element promote sealing when the device is closed. The device is also not exposed to torsion, because the flat surfaces of all the elements are supported on each other. Also if the device falls, it will only become more tightly closed, irrespective of the attitude in which it falls, while neither the flat surfaces nor the components of the sensitive internal element will be exposed to damage due to an external impact.

Placing the electronics required by the device is a separate body component also brings the advantage that the case parts of the device can be entirely detached form the body part, allowing them to be easily changed. This permits the appearance of the device to be personalized to suit the likes and needs of the user. Thus the health-monitoring device can be cost-effectively adapted to suit different user groups while using the same body part.

In the internal part of the device (or in the outer part, or in both) there may be a connector or connectors intended for data transfer, or other corresponding components, such as are generally found in digital devices. Examples of such components are displays, biometric identification devices, medicinal solutions (medicines, nanotechnologies), wireless and wired data-transmission means, memories and processor technologies, a microphone, a loudspeaker, and other user-interface elements. The device can also incorporate components adapted to personal health monitoring and care, additional devices and requisites, such as cleaning and hygienics devices, medicinal requisites, and storage spaces.

We use the term joint between the case parts to refer to the zone distinguished when observed from outside the device, which is formed between the case parts when they are fitted against each other. As there are no portions of the joint parallel to the direction of movement, it generally runs around the entire device

### BRIEF DESCRIPTION OF THE DRAWINGS

In the following, embodiments of the invention are described in greater detail with reference to the accompanying drawings. In the drawings

FIGS. 1a and 1b show side views of one embodiment of the device, closed and opened, correspondingly,

FIGS. 2a and 2b show cross-sections of a device according to one embodiment, closed and opened, correspondingly,

FIGS. 3a and 3b show cross-sections of a device according to one embodiment, closed and opened, correspondingly,

FIGS. 4a-4c show perspective views of one embodiment of the device opened, half opened, and opened, correspondingly,

FIGS. 5a-5c show perspective views of one embodiment of the device opened, half opened, and opened, correspondingly,

FIGS. 6a and 6b show side views of one embodiment of the device, closed and opened, correspondingly,

FIGS. 7a and 7b show side cross-sections of one embodiment of the device, closed and opened, correspondingly,

FIGS. 8a and 8b show cross-sections of the seal between the case parts, in the opened position, according to one embodiment,

FIG. 9 shows a perspective view of a device according to a third embodiment, equipped with means for measuring blood sugar,

FIGS. 10a-10c show a series of images of a health-monitoring device, in which casing according to one embodiment of the invention is utilized,

FIGS. 11a-11c show a series of images of a health-monitoring device, in which casing according to one embodiment of the invention is utilized,

FIGS. 12a-12d show a series of images of the implementation of the opening stages of the device, according to one embodiment,

FIGS. 13a and 13b show one further development of an embodiment of the device,

FIGS. 14a-14c show a second further development of an embodiment of the device,

FIGS. 15a and 15b show in greater detail the rail-slider arrangement of one embodiment of the device, and

FIGS. 16a and 16b show cross-sections of yet another further development of an embodiment.

### DETAILED DESCRIPTION OF THE PREFERRED EMBODIMENTS

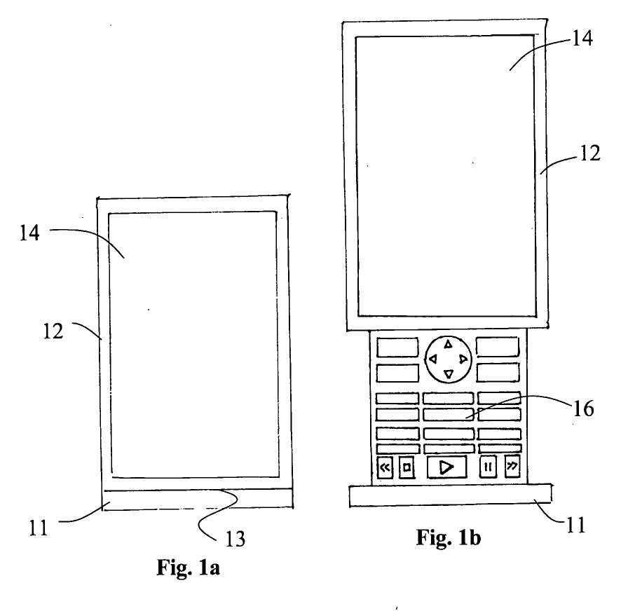

With reference to FIGS. 1a and 1b, according to one embodiment, the second case part 12 is a hollow (cavity-like) piece open at one side. The first case part 11 is arranged to move partly inside the second case part 12, in such a way that it runs through the open side of it and partly inside it. In the primary position (FIG. 1a), the first case part 11 and the second case part 12 rest against each other, so that an entirely or partly bevelled joint 13 is formed between them, at right-angles to the direction of movement. A keypad 16, which is thus tightly closed inside the case in the basic position, is in the first case part 11 and moves together with it. In the opened position, the keypad 16 is available for use. The display attached to the second case part 12 (or arranged to move with it) is marked with the reference number 14 in the embodiment of the figure.

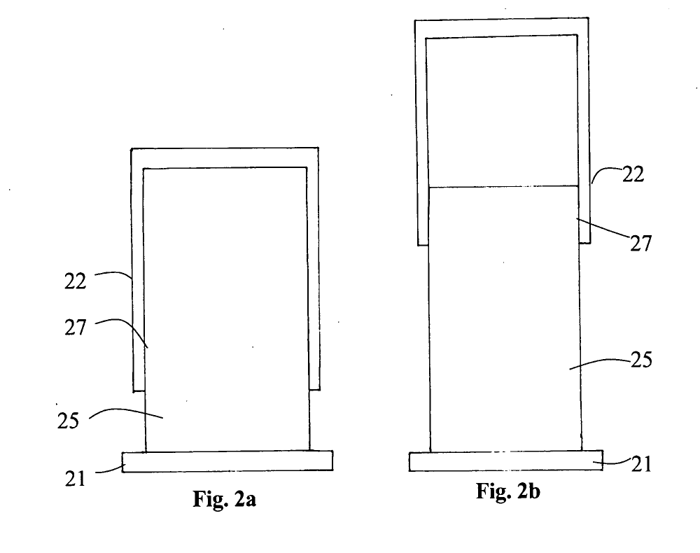

The part that remains visible outside the second case part 12 can comprise a detachable cover part. FIGS. 2a and 2b show one embodiment of the device according to FIG. 1 without the detachable cover and display of the second case part. Thus, the upper part forms a moving slider 22, which slides along rails or other carrier elements 27. A display (not shown) can be attached to the slider and takes up a position on top of the first case part when the device is closed.

Thus the slider 22 forms the internal part of the second case part (without a detachable cover). The display can be attached to the slider in such a way that, when the device is closed, the display takes up a position inside the body 25 of the first case part, or preferably on top of it, in which case the display can be read even when the device is closed. Once the slider has been slid into the opening position, the interface elements in the body 25 are exposed while the display can still be read attached to the slider 22. The slider 22 can be roughly of the inverted U shape shown in the figure.

The second case part of one preferred embodiment comprises an interchangeable outer cover, which can be attached to the slider 22, and through which the display area 34 is visible. This solution has the advantage that the sliding mechanism is protected and can be implemented with the highest possible quality, but the interchangeable covers can nevertheless be kept very simple and easily changed. This practice is also suitable for the three-part mechanism described later.

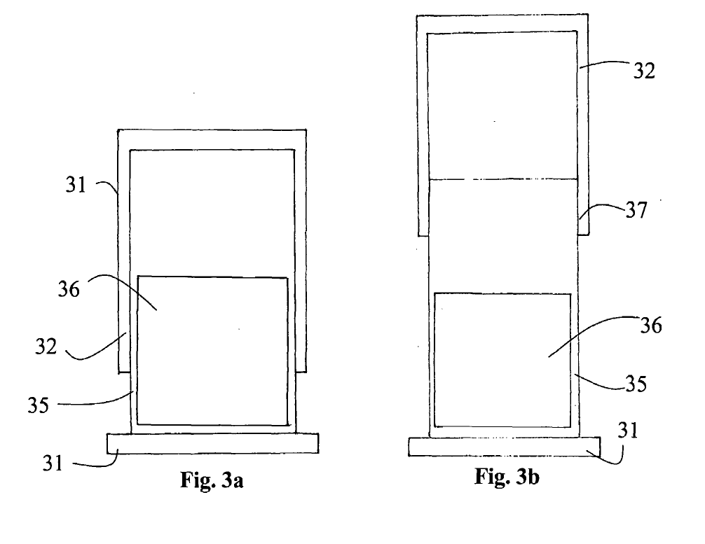

FIGS. 3a and 3b show an embodiment, in which there is also a key area 36 moving with the first case part 31. The key area 36 can further be divided into different portions, which open in steps, one at a time.

If electrical features are arranged in both case parts, which are generally user-interface elements, such as operation keys, display elements, or data-transfer or calculation units, the case parts will also be connected to each other electrically. This can be implemented, for example, in some way that is, as such, known.

The figures also show the counter surface in the internal part, against which the corresponding surface of the moving case part closes tightly. A separate seal, or separate seals can be attached to the mechanism, in order to close the case parts tightly against each other. The seals can be in an interchangeable outer cover, in the internal part of the device, or, for example, also as part of the slider.

The slider 22, 32 preferably moves relative to the first case part in such a way that its rail surfaces are essentially entirely inside the first case part, i.e. so that they cannot be accessed directly from outside the device, without detaching the case parts. Thus it is possible to further decrease the exposure of the sliding rails to the environment, even when the device is open. In addition, in the second case part is shaped to close tightly against the first case part. This shaping is formed by joints that are not parallel to the direction of movement. They can thus bevelled, at right angles to the movement, curved, or have combinations of these. Thus a compressive force is created in the joint when the device is being closed, which makes tight closing possible.

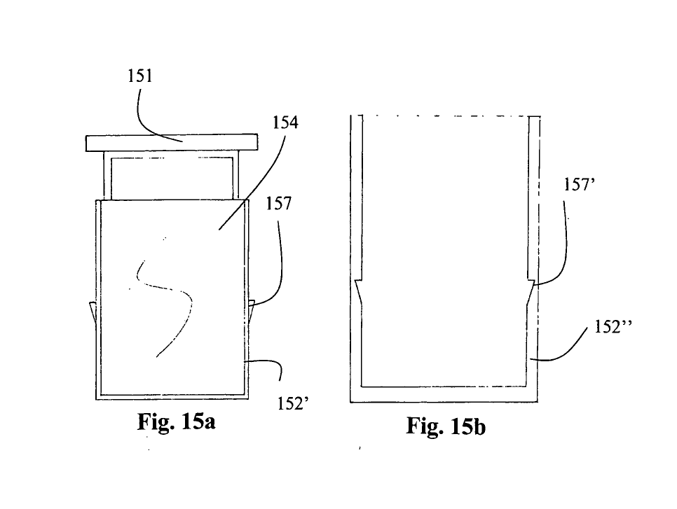

FIG. 15 shows by way of example the attachment of the display 154 to the slider 152 and the interchangeable outer cover 152′. For operation, there are rails in the device, along which the slider 152′ moves. The display 154 is attached to the slider 152′. An outer cover 152″, which covers the display 154 and the sliding mechanism, can be fitted in turn on top of this totality. The outer cover 152″ closes tightly against the first case part 151. The figure also shows one way to create the attachment of the outer cover, with the aid of teeth 157 and notches 157′.

Other implementations too, in which the second case part does not comprise a separate slider and cover, but in which the case part slides directly along ‘grooves’ or similar in the device, come within the scope of the present inventive idea. The slider implementation shown permits, however, very low siding friction and reduces the ‘play’ between the components. This achieves a well-controlled and precise movement and a simple possibility to change covers. The slider implementation is also relatively cheap.

The display, or other moving electronic modules can be electrically connected to the rest of the electronics of the device using mechanisms known from existing slider phones, or similar. At its simplest, a flexible guide is suitable for this task. Once the cover is on top of the device, the connection of its display too will remain well protected from the environment.

A combination is particularly advantageous, in which the display is in the second case part and visible on top of the first part through the second case part when the device is in the primary state, the operation keys of the first part being exposed from under the cover of the display/second case part when the device is opened and the device closes tightly into its primary position with the aid of a dividing joint deviating from the direction of movement.

The display can also be installed on the outer surface of the second case part. If there are interchangeable outer covers in the device, they can have a transparent part or opening in them, which is dimensioned in such a way that it closes relatively tightly around the display attached to the slider. The interchangeable cover of the second case part can also be in two parts, i.e. it has a front part and a separate back part. All the case and body parts can also contain several interchangeable covers or cover components (the interchangeable covers can be of several parts). This also applied to embodiments with three or more parts. The various operational modules can also be changed as parts of the cover, or independently of them.

Generally, applications, in which the display moves attached to the slider along with the second case part, achieve the advantage that a large operational surface is created, both when the device is closed and when it is opened, but the outermost surface of the second case part can also be ‘dumb’, i.e. for instance an interchangeable plastic cover. This brings a further advantage, for example, if the cover is scratched or when personalizing the device. The display and cover on top of each other will, however, take up space, so that in interchangeable covers there can be an opening the size of the display and the display comes directly to the outer surface.

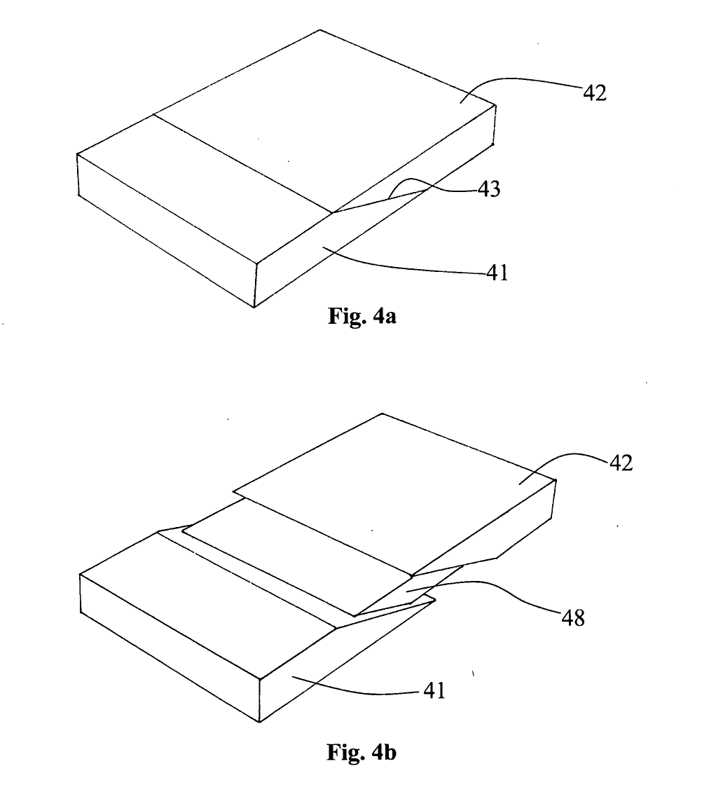

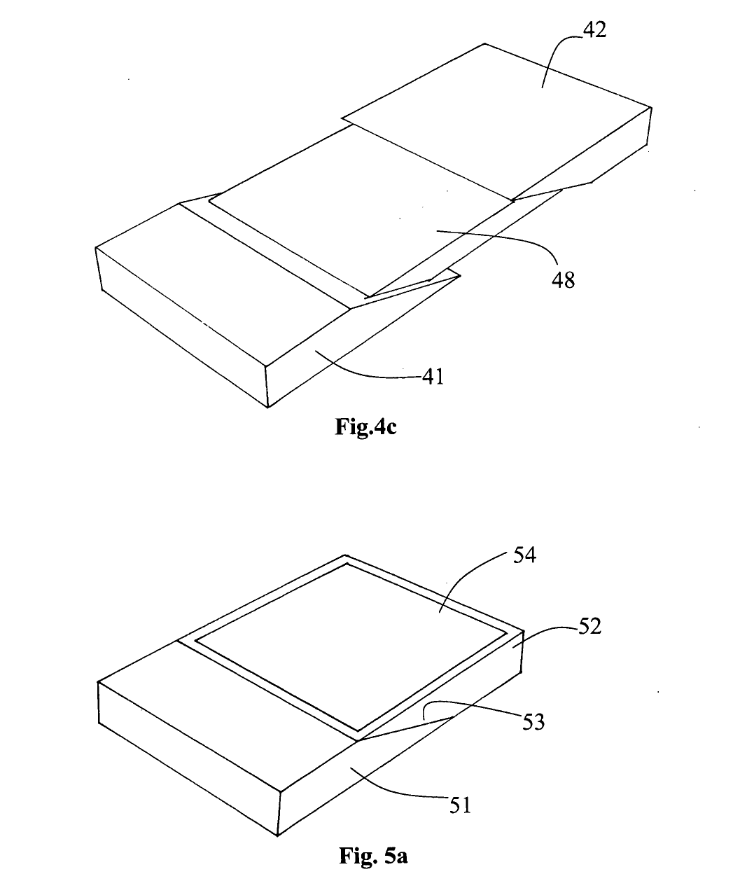

With reference next to FIGS. 4a-4c, the device can comprise three parts that move relative to each other. The case parts 41 and 42 form the outer surface of the device when it is closed, when a joint 43 that is comparatively tight and deviates from the direction of movement is formed between the parts. The case parts 41 and 42 are not connected directly to each other, instead a body part 48 is fitted between them. Each case part can be slid separately relative to the body part 48, thus naturally also achieving a sliding movement between the case parts 41 and 42.

The case parts 41 and 42 are essentially hollow (cavity-like), pieces that are open on one side, so that the body part 48 is able to slide inside them in one dimension, and then out. When opening, the sliding movement is generally stopped by end-stops fitted to the sliding means. Particularly the passive case parts can also entirely detachable form the body part, making it possible to change and personalize them. The case parts can also comprise a base part (compare with the aforementioned slider mechanism), arranged to move relative to the body part, and a detachable cover part.

In this embodiment too, the movement between the parts can be implemented with rails, which remain essentially entirely concealed when the device is closed. In the open position too, the rails of the case parts, or similar can be protected, for example, inside the body part 48. Alternatively, the outer surface of the body can itself be suitable for sliding relative to the base parts, so that separate rail components will not necessary be required. For example, the essentially rectangular geometry illustrated by the figures can be easily implemented in terms both of sliding and sealing.

According to one preferred embodiment, which is also illustrated in FIGS. 4a-4c, the movement between the case parts has at least three positions. This means that each case part 41, 42 moves and ‘locks’ separately, relative to the body part 48. This is preferably implemented in such a way that, in order to move, or start the movement of the case parts 41, 42, different magnitudes of force relative to the body part 48 are required, or that one or other case part is entirely locked relative to the body part 48. Thus, when the device is initially in a closed state (FIG. 4a) and the user begins to pull the case parts 41 and 42 relative to each other, first of all sliding takes place, between one case part (for example, 41) and the body part 48, to the operating position (FIG. 4b) and only after that does sliding take place between the other case part (for example, 42) and the body part 48 to the operating position.

The phasing of the opening can also be implemented by means of a mechanism, which releases the second case part to open only once the first case parts is entirely opened. In the same way, the closing can be phased, in which case the first part cannot close until the second part is entirely closed.

The bevelled dividing joint 43 illustrated by FIGS. 4a-4c permits not only a good seal, but also makes it possible to operate the device with one hand, in such a way that the thumb moves one case part and the other fingers the other part. With the aid of the bevelled dividing joint a sufficiently long movement can be obtained with one hand.

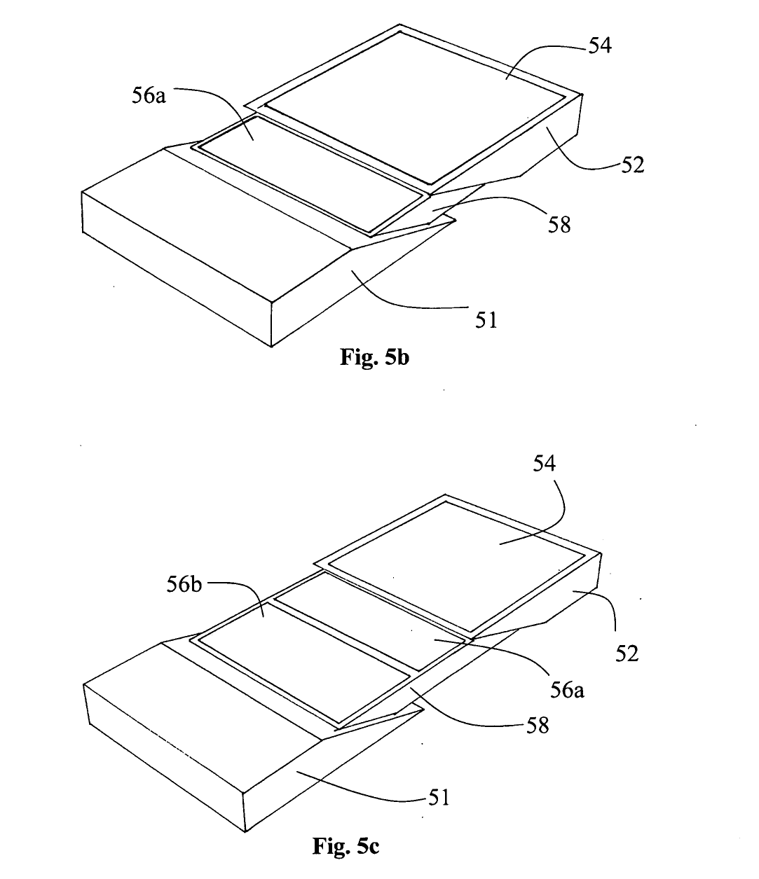

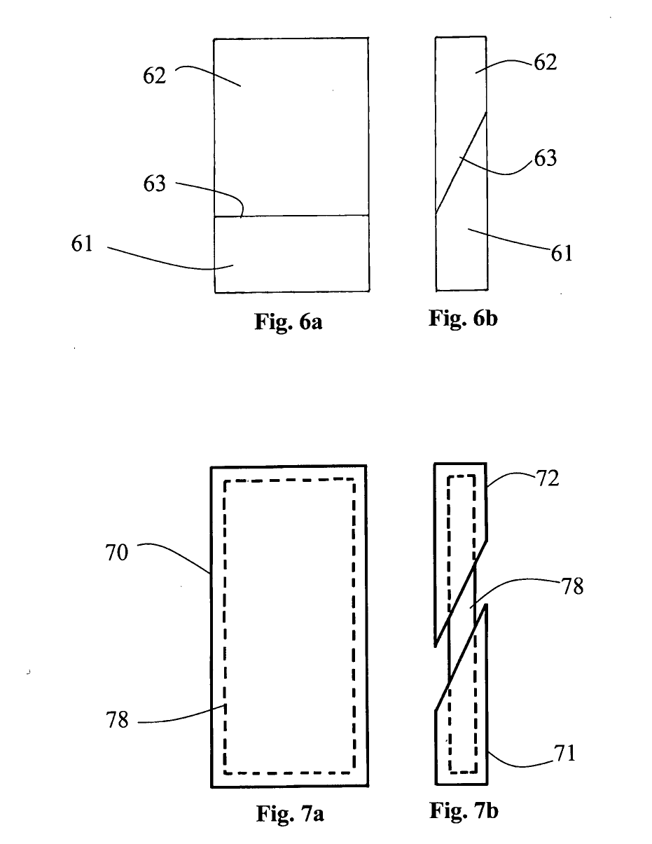

FIGS. 5a-5c illustrate a three-part mechanism like that of FIGS. 4a-4c, in which a display 54 is attached to the second case part 52, in such a way that it can be seen when the device is closed. In the body part, there are in addition first and second key areas, 56a and 56b, correspondingly, which are exposed one at a time with the aid of the stepped movement of the case parts. One skilled in the art will understand that the mechanism also permits the display and key areas to be placed in some other manner. For example, one can refer to an embodiment, in which the display is in the body part, which is exposed in the first stage of the opening, and a keypad, which is exposed in the second stage. Another example is an embodiment, in which there is a keypad in the outer surface of the first case part, a display in the second case part, and further an additional keypad and/or additional display in the body part.

According to one embodiment, the device comprises means for showing still images and/or video images on the display.

A particularly preferred embodiment is a solution, in which the first and second body parts are manufactured from a transparent material on at least one side, so that a display combined with the body part, or a touch-sensitive display can be seen through the body part, even when the device is mechanically closed. In this embodiment there is no need for a moving display, so that for this part the device is inexpensive.

Instead of/in addition to the display and keypad, in the device there can also be, for example, a touch-sensitive display, which is given different operations depending on the operating state of the device, a camera, a light, a microphone, a loudspeaker, connectors (electrical, optical), space for a battery, space for a SIM card (or similar), space for memory media (such as a memory card, a hard disk, or digital media). Instead of/in addition to electrical operations, elements, or modules, in the device there can also be mechanical operations, such as mechanical switches (e.g., for detaching the covers), or other operations, as will later be described in greater detail with the aid of an example illustrating a personal health-monitoring device. The operations, elements, and modules can be situated as required on the front or rear surfaces of the device, on the side, or on the ends, on the outer surface, or in the body part, i.e. in the internal parts of the device. The operations, elements, and modules can also be moved to different positions (for example, changing the imaging direction of the camera).

Operations, such as answering a phone call, activating the display/microphone, starting/stopping media playing, etc., can be linked operationally to the different stages of the sliding.

Generally, a three-part mechanism has the advantage that the joint of the device is brought closer to the centre of the device while the outer shell sliding in both directions permits symmetry and the opening of the device farther than the sliding of only one half. Placing the joint more in the centre of the device is an important usability factor. Particularly if the joint placed in the centre of the device is given a bevelled form, as in FIGS. 4 and 5, good usability will be achieved (palm-thumb movement). However, this solution is still tight, because a compressive forces acts on its joint when the device is closed.

According to one embodiment, in the closed position the outer surface of the device is free of all the user-interface elements. It is then impossible to say what the device is simply by looking at it.

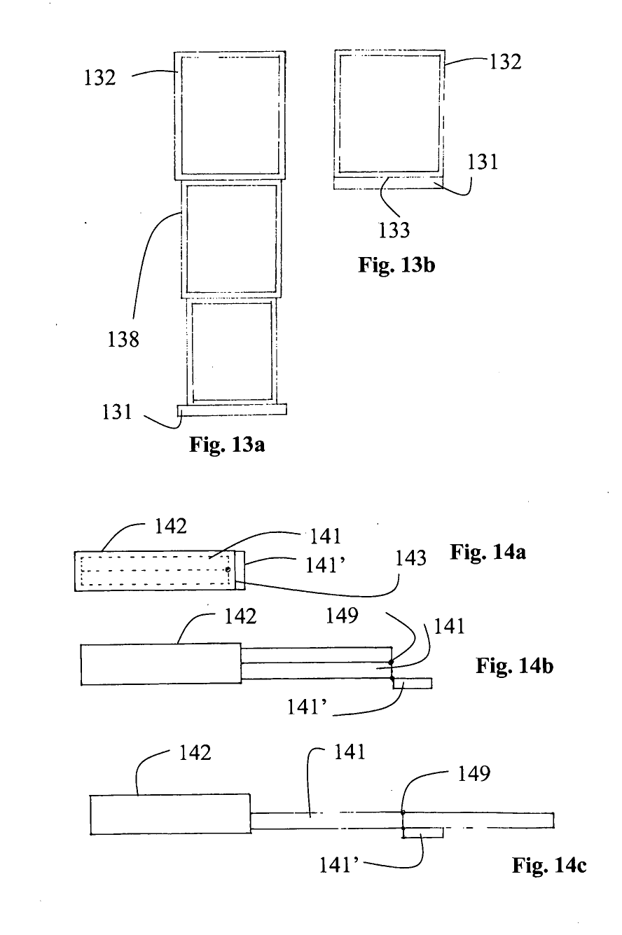

The device can also be implemented in such a way that in it there are several parts set inside each other (telescopically opening device), in which case the surface area of the device will be even greater when it is opened. Thus it will be possible to implement, for example, a fairly large keypad. This is illustrated in FIG. 13. In the parts to be revealed from inside the device there can also be additional parts opening around a pivot (clamshell-like), as illustrated in FIG. 14. The additional parts according to the above description can also be in the uppermost part of the device, as well as in the various parts of the three-part mechanism.

In the three-part implementation described above, the first and second case parts preferably comprise a slider connected to rails fitted to the body part, and covers, preferably interchangeable covers. The electrical modules possibly located in the body part are then attached to the slider. Thus in a three-part solution too, the outer covers can be interchangeable, in the same way as in a two-part implementation.

The sliding rails and other joints of the device, such as the dividing joint 43, 53 are preferably implemented with a degree of tightness comparable to that of existing devices, so that the device, even when opened, will not be unreasonably exposed to the environment. In addition to a direction differing to the direction of movement, there can also be other features in the dividing joint that will promote tightness, such as overlaps between the case parts, or elastic sealing elements.

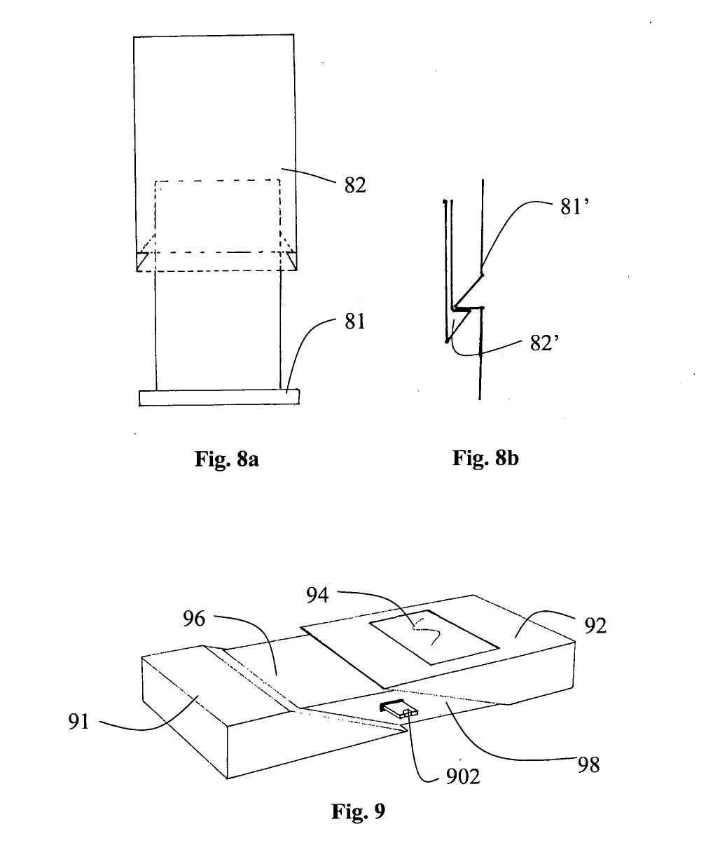

With reference to FIGS. 8a and 8b, it is also possible that in the opened position the case parts 81, 82 that move relative to each other press tightly against each other, so that a particularly tight joint is created, even when the device is open. The direction of even such a joint preferably differs from the direction of travel over the entire distance of the joint. The first case part 81 comprises a counter-piece 81′, against which a protrusion 82′ from the second case part 82, or similar presses. The same arrangement is used to restrict the movement between the case parts. FIG. 8b shows the left-hand edge of the device of FIG. 8a in greater detail. A corresponding joint can run around the entire boundary zone between the case parts, i.e. it can also be located on the front and rear sides, so that there is no access for dirt to the internal part delimited by the case parts, even when the device is open. A corresponding arrangement can be implement between the case parts and the body in connection with a three-part device solution too.

As briefly stated above, the opening or closing movement of the cases can also trigger or expose various elements for further operations. With reference to FIG. 9, which shows a new type of portable blood-sugar meter, the movement can be used, for example, to trigger and/or exposes a needle device (also ‘lancet device’, not shown), and bring out a sensor strip 902 used for measuring blood sugar. The needle device and sensor strip 902 are preferably contained in the body part 98 of the device. The first and second case parts 91, 92 preferably not only trigger and/or bring out the means in question, but also reveal user-interface components for personal (and repeated) blood-sugar monitoring. In the figure, the idea is applied to a three-part mechanism, but can also be applied to a two-part mechanism.

Due to their tight seal, the casing solutions according to the various embodiments of the invention are particularly well suited for use in personal health-monitoring devices. Besides measuring blood sugar, the invention can also be applied in connection with devices and methods intended for the physical, chemical, or medical analysis of tissue fluids, other body fluids, and other samples, such as biopsy specimens. In particular, the health-monitoring device with a shutter element, disclosed in FI patent application 20050768, can be fitted into the present casing, in such a way that the first and/or second case part depicted here acts as the shutter element, and the first second or first case, correspondingly, or the body part (in a three-part mechanism) forms the body of the device. The invention can also be applied to non-invasive forms of measurement and care, which can comprise, for example, radiation sources and receivers.

The device according to the invention can also contain other requisites relating to personal health care, such as cleaning cloths and agents, reserve nutrition, medicines, and medical devices.

In the outer or inner part of the interchangeable cover, or in connection with some other body or case part, there can be storage spaces, for example, for the disposal parts of the meter, such as sensor strips and lancets, as well as other accessories. Parts and components can be taken for use from such spaces, or advantageously charge or change components in the meter manually or automatically, for example, in connection with changing the covers.

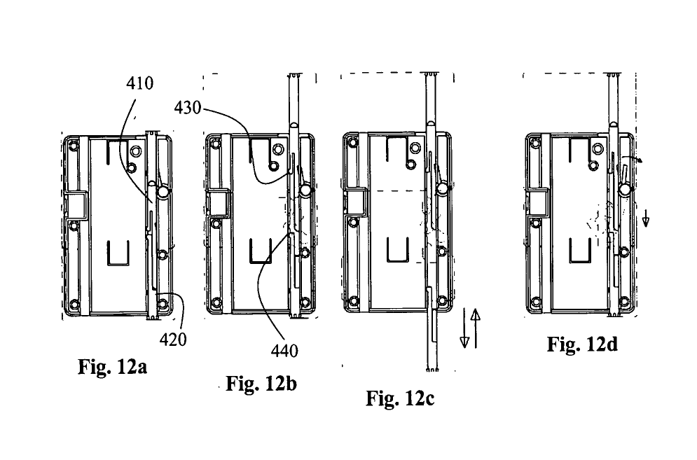

FIG. 10 shows a health-monitoring device comprising three-part casing in greater detail, in which the first and second case parts form a two-part shut-off element, by means of which the basic operations of the device are activated. The first and second shutter elements 210 and 220 slide separately relative to the body 230. Operation of the device starts (FIG. 10b) by moving the first shutter part 210, when the operating area of the body 230 becomes visible and a new strip 258 is pushed out from the side of the device. The lancet can be triggered by the movement of the second shutter part (FIG. 12c) and then be released by the shutter element, or by a separate release element. Reference number 266 marks the strip-cutting switch. When the first shut-off part 210 is moved farther relative to the body 230 (FIG. 10e), the strip roll 252 is exposed and can then be changed in this position. The lancet typically protrudes like the strip from the opposite side of the flat device.

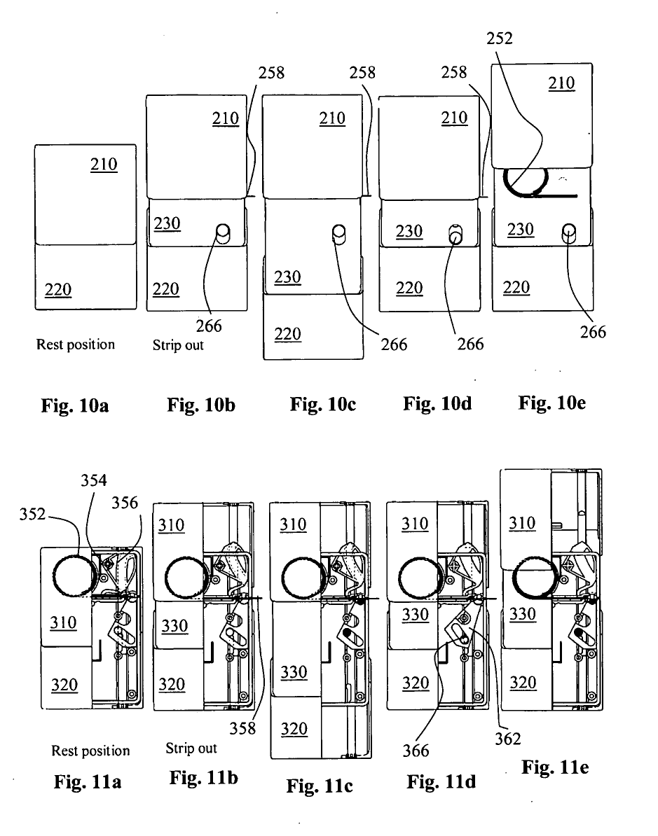

FIG. 11 shows greater detail of an example of a mechanism for achieving the operations described above. When the first shut-off part 310 is pulled, the feed element 356 pushes one strip 358 out of the side of the device, after which the strip 352, which is in a roll, rotates for a corresponding distance. In this case, the feed element is pivoted to the body 330 of the device by a pivot 354. The effect of the strip cutting switch 366 is created by a cutter 362 that is operationally connected to it. Here the cutter is rotatably attached to the body 330. When cutting, the cutting head of the cutter 362 travels close to the fixed counter parts of the body and together these cut through the strip 352, rather like scissors.

According to a preferred embodiment, the device comprises a container portion/vessel, which can be tightly closed, even when the device is in the second position. Such a vessel typically contains analysis apparatus, which must be kept extremely clean, or closed from the air. In the case of a blood-sugar measuring device, the measuring strips can be located in this container. The container can be opened for only the time needed with the aid of a mechanical or electrical interface element, when the device is in the second position. The container is preferably located most typically in the body part of a three-part device solution.

FIG. 12 shows a mechanism, by means of which a phased movement can be achieved in the case of three-part casing. The first and second case parts are attached to rods, or to other suitable phasing elements 410 and 420, correspondingly, which comprise sprung teeth 430 and 440, or corresponding means for locking their position relative to the body (FIG. 12a). Once the rods overlap in the first position, the teeth 440 cannot escape from the notches, so that the second case part cannot slide relative to the body. The first shut-off part, however, can be slid to the operating position (FIG. 12b), when the movement also releases the tooth 430 and thus permits the movement of the second body part (FIG. 12c). The case can be closed in the opposite sequence (FIG. 12d).

In a three-part implementation, the outer case parts can move symmetrically relative to the body part, which will permit the device to be used from both sides in the same way. Various operations can be linked to the body part and the top, bottom, and side surfaces of the body part. Particular mention should be made of the embodiment illustrated by FIG. 9, which includes some mechanism or electrical user-interface on a side surface, or both side surfaces of the body part, beneath the bevelled joint part. It is always possible to expose operations located on different sides with completely the same movement, simply by turning the device in the hand. The movements of the case parts can also be divided to operate in sequence.

Typically, the sliding movement of the embodiments described above is created in both directions with the aid of a force produced by the user, though various, as such known, spring mechanisms and corresponding mechanical operations can be added to the casing. Stated in general terms, the device preferably comprises means for locking the first and second case parts relative to each other in a first and, on the other hand, in a second position (or in several second positions), in which case, the force required to move them away from the position in question, will be greater than that required to move them between the various positions. Various electrically operated, or electrically aided opening and/or closing operations are also possible.

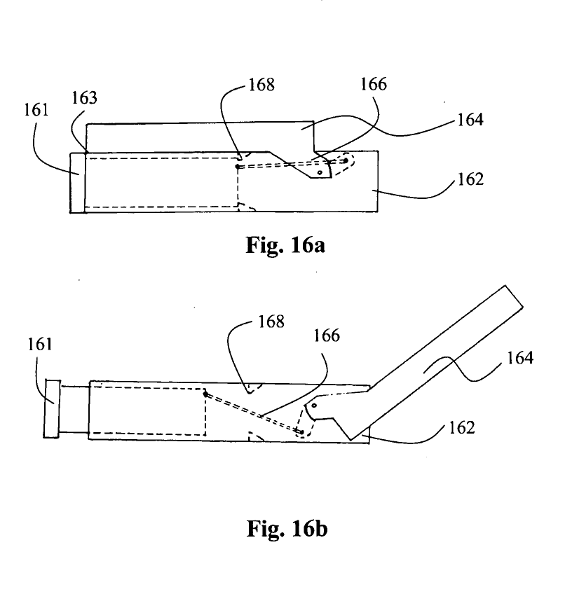

In addition, the electrically operated or preferably mechanical elements can direct a force on at least one joint of the device, when the joint in question is closed, which presses the parts forming the joint together, in order to improve the seal. An example of such an arrangement is shown in FIG. 16, which is described in greater detail later. In addition to this, the force can be produced by, for example, internal coil springs, or other methods obvious to one skilled in the art.

FIGS. 13a and 13b illustrate a more highly developed embodiment. It consists of three parts that slide telescopically relative to each other. Unlike in the mechanisms described above, the first and second case parts 131, 132 are arranged to slide relative to the body part 138, in such a way that, in the first position, the joint 133 is formed essentially in the side area of the device (in the sliding direction). In addition, the first case part 131 is arranged to slide inside the body part, so that it does not entirely cover (surround) the body part 138, unlike the solution illustrated by FIG. 4. In the first case part there is a shoulder, which in the first position is arranged to press tightly against the second case part. The location and shape of the joint can naturally vary in solutions of this kind too.

FIGS. 14a-14c illustrate a further embodiment, in which there is a three-part first case part 141. It comprises a cover 141′, which is arranged to lie tightly against the second case part 142. When the cover is opened, typically by turning it relative to a pivot arranged in the first or second case part, the first case part 141 can be slid out from inside the second case part 142. Further, when the device is in the second position, the two halves of the first case part 141 can be opened by rotating them open relative to a pivot 140 (compare a so-called clamshell phone). The solution offers not only a large surface area for locating the user-interface, but also a tight casing in the closed position, for example, for transporting the device. A display can be located, for example, in the second case-part 142, in the first case part 141, or in both.

FIGS. 16a and 16b show, in turn, an embodiment, which comprises a third case part 164 connected operationally to a first 161 and a second 162 case part, and which is arranged to move between at least two different positions (Figures a and b, correspondingly), in such a way that, in one position the first 161 and second 162 case parts lock against each other, forming a tight joint 163. This can be achieved, for example, in such a way that the third case part 164 is rotatably pivoted to the second case part 162, and the device comprises means 166 for changing the rotational movement of the third case part 164 into a sliding movement of the first case part 161 relative to the second case part 162. The means 166 can comprise, for example, of a rigid or elastic elongated element, which at one end is pivoted to the third case part 164 and at the other end to the first case part 161. The rotational movement and the means 166 are preferably arranged in such a way that, when the third case part 164 is in the closed position (the first position of the device), the first 161 and second 162 case parts are continuously pressed against each other, and the third case part 164 is pressed onto the second case part 162. The Figure also shows a protrusion 168, which can be fitted to the inner surface of the second case part 162, in such a way that a tight joint is created between the rear edge of the first case part 161 and the second case part.

Particularly in this embodiment it is advantageous to use a seal in the seam 163, preferably a seal that is to some extent flexible (elastic). When the flap 164 is closed, when the first and second case parts 161, 162 meet in the joint area 163, the spring force of the seal will resist closing. At a certain stage (generally when the element 166 connecting the flap 164 and the first case part move over the pivot of the flap 164), the direction of the force changes. The spring force of the seal then keeps the flap 164 closed and the joint 163 tight.

A corresponding mechanism can also be located on the outside of the device, connected to the visible outer surface of the first case part 161. There is then no need for additional components in the sealed internal area. As one skilled in the art will understand, such a geometrical spring-assisted locking can be associated with any of the other embodiments.

A spring-assisted mechanism can also be installed inside the device, as shown, for example, in publication JP2004253526. The operation of the spring mechanism will then cause the lock components of the device to move, and no separate operating element will be needed. The spring mechanism can be installed in such a way that it presses the joint closed when the joint is shut, thus improving the seal of the device.

The third case part too can comprise at least one user-interface element.

Spring mechanisms, like those described above, can also be use in a three-part embodiment.

Sliding rails permitting the sliding movement required by the embodiments described above, or corresponding elements permitting sliding can be implemented in some manner that is, as such, known. For example, US patent publication 2005/261042 discloses a rail mechanism, which is cheap and easy to install, and which can be applied to the present device.

Health-monitoring devices comprise a data-processing unit, which, in a two-part case, is generally located in the first case part and, in a three-part case, generally in the body part (i.e. generally in the case part that forms the inner unit of the device in the closed position).

A casing like that described above can also be manufactured as a separate accessory. Thus, the case can also be used to protect existing goods and devices, for example, from impacts, dust, and moisture. As a separate protector for health-monitoring devices, it could replace existing soft plastic or leather cases, in which case it will also protect the device in rough use.

The embodiments described above are by way of examples, and can be combined and adapted freely within the scope of the inventive idea disclosed in the Claims. The Claims must be interpreted to their full extent and taking the equivalence interpretation into account. Thus the term ‘health-monitoring device’ includes not only devices, particularly personal devices, intended for health monitoring and health care, but also, for example, for biometric identification and those intended for the making of measurements relating to sport. These latter devices are, for example, devices intended for measuring lactic-acid content. Thus, ‘health-monitoring device’ also includes separate personal biological analysis devices.

While the present invention has been illustrated and described with respect to a particular embodiment thereof, it should be appreciated by those of ordinary skill in the art that various modifications to this invention may be made without departing from the spirit and scope of the present invention.

1-46. (canceled)

47. A portable personal body fluid measuring device, which comprises:
at least one health-monitoring element for measuring body fluid, and
a first case part and a second case part, which are arranged to slide in at least one direction relative to each other from a first position into a second position in order to expose said health-monitoring element,
wherein
the first and second case part are arranged in the first position to lie tightly against each other to protect said health-monitoring element, so that a joint if formed between them, and
the direction of the said joint deviates over its entire length essentially from the said direction of sliding, in such a way that the joint is continuous and forms a closed path that runs around the entire device on the outer surface of the body fluid measuring device.

48. The body fluid measuring device according to claim 47, wherein the said joint comprises at least one zone, the direction of which is essentially at an angle to the said direction of sliding, deviating from it typically by about 10-80°, preferably by about 30-60°, in order to cause a force component in the joint, at right angles to the direction of sliding, when the first and second case parts are pressed together.

49. The body fluid measuring device according to claim 47, wherein the joint is located entirely essentially on a plane at an angle to the direction of sliding and essentially half way, when examined in the direction of sliding.

50. The body fluid measuring device according to claim 47, wherein the sliding movement between the first and second case parts is stepped.

51. The body fluid measuring device according to claim 47, wherein the sliding movement between the first and second position, or one or more parts of the movement is arranged to achieve one or more electrical or mechanical operations in the body fluid measuring device.

52. The body fluid measuring device according to claim 47, wherein said health-monitoring element comprises a blood sugar measuring strip.

53. The body fluid measuring device according to claim 47, wherein said health-monitoring element comprises skin-piercing means.

54. The body fluid measuring device according to claim 47, wherein the first case part and the second case part are each separately connected in a sliding manner to a body part, so that the body part is arranged to move into both the first and the second case part.

55. The body fluid measuring device according to claim 47, wherein the movement between the first and second case parts is implemented using rails.

56. The body fluid measuring device according to claim 54, wherein in the first position the first and second case parts cover every side of the body part essentially entirely.

57. The body fluid measuring device according to claim 54, wherein the first and second case parts are connected to the body part in a sliding manner, in such a way that, in order to slide them relative to the body part, forces of substantially different magnitudes are required.

58. The body fluid measuring device according to claim 54, wherein the body part contains at least one user-interface or health-monitoring element of the device, which the first and/or second case parts cover in the first position and which is arranged to be revealed by sliding the first and/or second case parts.

59. The body fluid measuring device according to claim 54, wherein rails are fitted to the body part and the first and/or second body parts comprise:
a slider, which is arranged to slide along the said rails,
a cover, which is attached to the slider detachably, or which can be attached to it,
and the said case part is arranged to lie tightly against the other case part of the device in the first position.

60. The body fluid measuring device according to claim 54, wherein the slider is equipped with a display, which is arranged to be visible through the said cover.

61. The body fluid measuring device according to claim 47, comprising a keypad, which is arranged to be covered in the first position.

62. The body fluid measuring device according to claim 47, comprising a display, which is arranged to be covered mechanically in the first position and to be exposed in the second position.

63. The body fluid measuring device according to claim 47, wherein the second case part is arranged to be transparent at least at the location of the display, in such a way that the display can be read in the first position.

64. The body fluid measuring device according to claim 47, wherein the display is attached to the second case part.

65. The body fluid measuring device according to claim 47, wherein the health-monitoring element comprises means for analysing a body fluid, or other physiological, chemical, or biological component or property, such as blood, in which case the means are arranged to be covered in the first position and to be exposed with the aid of a movement between the first and second case parts.

66. The body fluid measuring device according to claim 65, wherein the means for analysing a body fluid are arranged to be ready to measure with the aid of said movement between the first and second case parts.

67. The body fluid measuring device according to claim 47, comprising:
a skin-piercing means, such as a triggerable lancet and
blood-sample measuring means, such as blood-sugar measuring means,
particularly protruding measuring strips, for determining the analysis of blood
sugar or similar, from a drop of blood produced with the aid of the skin-piercing means.

68. The body fluid measuring device according to claim 67, wherein both the skin-piercing means and the measuring strips are protected with the aid of the said case parts when the device is in the first position.

69. The body fluid measuring device according to claim 67, wherein the skin-piercing means and the blood-sample measuring means are located on the sides of the body part.

70. The body fluid measuring device according to claim 47, wherein the case parts can be easily changed.

71. The body fluid measuring device according to claim 47, comprising means for moving from the first position to the second with the aid of a spring force or similar, and a release element for starting this movement.

72. The body fluid measuring device according to claim 47, wherein the first and/or second case parts can be further extended with the aid of a sliding or pivoting mechanism, when the device is in the second position, in which case the sliding or pivoting mechanism in question is essentially entirely protected inside the case parts, when the device is in the first position.

73. The body fluid measuring device according to claim 54, wherein, when the device is in the second position, at least the second case part presses against the body part, in such a way that a tight joint is formed between them, the direction of which differs over its entire length essentially from the said sliding direction.

74. The body fluid measuring device according to claim 47, wherein an elastic seal is fitted to the first and/or second case part, in order to further seal the joint.

75. The body fluid measuring device according to claim 47, comprising a spring mechanism, which creates a force that holds the case parts closed.

76. The body fluid measuring device according to claim 47, comprising a container portion, which can be separately opened and tightly closed in any position of the case parts, the container portion preferably comprising analysis apparatus, such as blood-sugar measuring strips.

77. The body fluid measuring device according to claim 47, wherein the opening movement is arranged to take place in several parts.

78. The body fluid measuring device according to claim 47, the device being a blood-sugar measuring device.

79. The body fluid measuring device according to claim 47, comprising one or more electronic components.

80. The body fluid measuring device according to claim 79, wherein the electronic component is a display, a biometric identification means, a medicinal solution, a wired or wireless data-transfer means, a memory component, a processing component, a cleaning or hygiene means, a microphone, a loudspeaker, or a corresponding user-interface element.
